(19) United States
(12) Patent Application Publication
(10) Pub. No.: US 2016/0242990 A1
(43) Pub. Date: Aug. 25, 2016

(54) Heated Simulated Rock For Massage Therapeutic Use

(71) Applicant: Justin Kiril Demski, SR., Las Vegas, NV (US)

(72) Inventor: Justin Kiril Demski, SR., Las Vegas, NV (US)

(21) Appl. No.: 13/999,805

(22) Filed: Mar. 21, 2014

(51) Int. Cl. A61H 7/00; A61F 7/00

(52) U.S. Cl. CPC A61H 7/003; A61F 7/007; A61H 2201/0207; A61H 2201/0228; A61F 2007/0071; A61F 2007/0078; A61H 2201/0153

(57) ABSTRACT

A simulated rock includes a heated portion and a self-contained rechargeable battery operated heater. Two or more resistors embedded in the lower portion of said rock heats the lower portion while keeping the larger upper portion cool to the touch.

### CROSS REFERENCE TO RELATED APPLICATIONS

N/A

### STATEMENT REGARDING FEDERALLY SPONSORED RESEARCH OR DEVELOPMENT

N/A

### REFERENCE TO SEQUENCE LISTING, A TABLE OR A COMPUTER PROGRAM LISTING COMPACT DISC APPENDIX

N/A

### BACKGROUND OF THE INVENTION

The present invention relates to a heated simulated rock for massage therapeutic use. Currently, hot rock massage is performed by a licensed massage therapist by means of heating smooth river stones in a bath of hot water heated to between 120 degrees Fahrenheit to 140 degrees Fahrenheit. The heated smooth river stones are then held in the massage therapists hands and continuously moved across a clients back and limbs to therapeutic effect. While the effect to a client is therapeutic, the effect on the hands of the therapist is not. Complaints ranging from uncomfortably to scalding of the therapists hands have been reported from using heated river stones in this way. The Applicants device is used to eliminate this problem. Applicant is unaware of any previous art that addresses this issue.

The object of the invention is to provide a simulated rock which is heated to between 120 degrees Fahrenheit and 140 degrees Fahrenheit on the one side being moved across a client's back and limbs, while the side held by the massage therapist remains comfortably cool.

The Applicant's invention will also remove the need for a heating device to hold and heat the water that the current river rocks require in order to be heated, as well as eliminating the need to pause the massage to obtain new hot stones when the first stones used become cooled.

### BRIEF SUMMARY OF THE INVENTION

The present invention relates to a heated simulated rock for massage therapeutic use. The present invention includes the following interrelated objects, aspects and features:

(A) In a first aspect, the invention includes a handheld portion and a lower portion to be heated.

(B) The upper, or handheld, portion is a hollow chamber which contains custom batteries, a three position switch, a light emitting diode, a plug to recharge the batteries as needed, and the necessary wires to connect them all. Four wires from the upper, or handheld, portion of the rock will be connected to two resistors embedded in the lower portion to be heated to simulate a heated river stone when used on the back and limbs of a client.

(C) The inventive simulated rock surface will be made of sturdy Cone 10 porcelain slip poured from a mold designed to allow for the insertion of all components listed in (B) above. In the preferred embodiment, the outer surface of the simulated rock is waterproofed and may be decorated in different colors and/or designs to render the simulated rock aesthetically pleasing.

These and other objects, aspects and features of the present invention will be better understood from the following detailed description of the preferred embodiment when read in conjunction with the appended DRAWING FIGURES.

### BRIEF DESCRIPTION OF THE DRAWINGS

FIG. 1 shows a perspective view of the present invention and the imperative components as relating to the inventions method of heating.

FIG. 2 shows a perspective view of the present invention and the imperative components as relating to the inventions method of recharging its batteries.

FIG. 3 shows a schematic representation of the electrical circuitry of the present invention.

FIG. 4 shows a detail of the waterproofed aspect of the present invention.

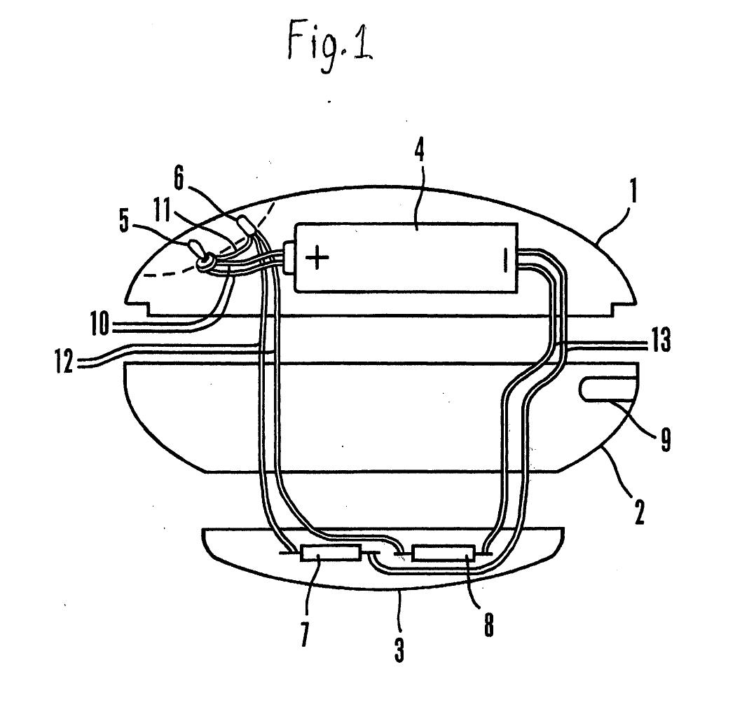

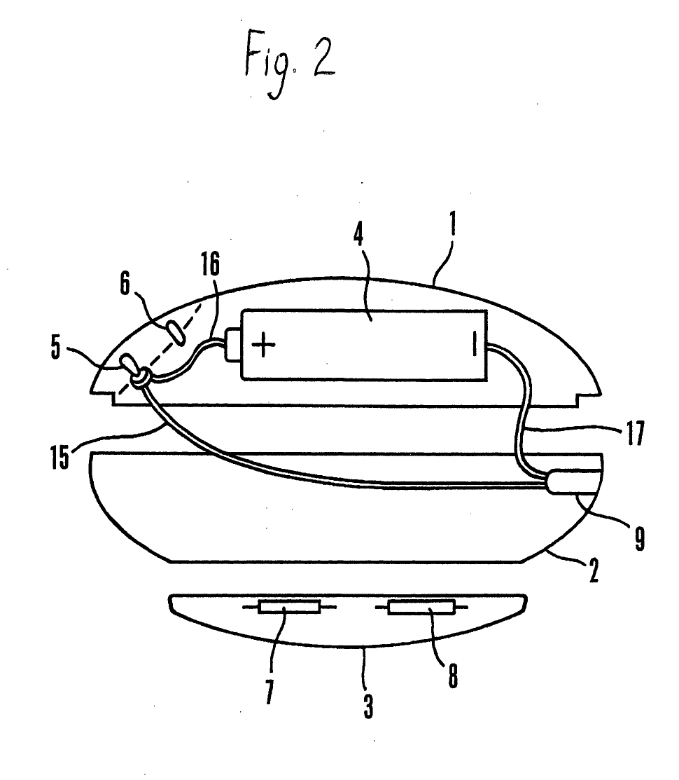

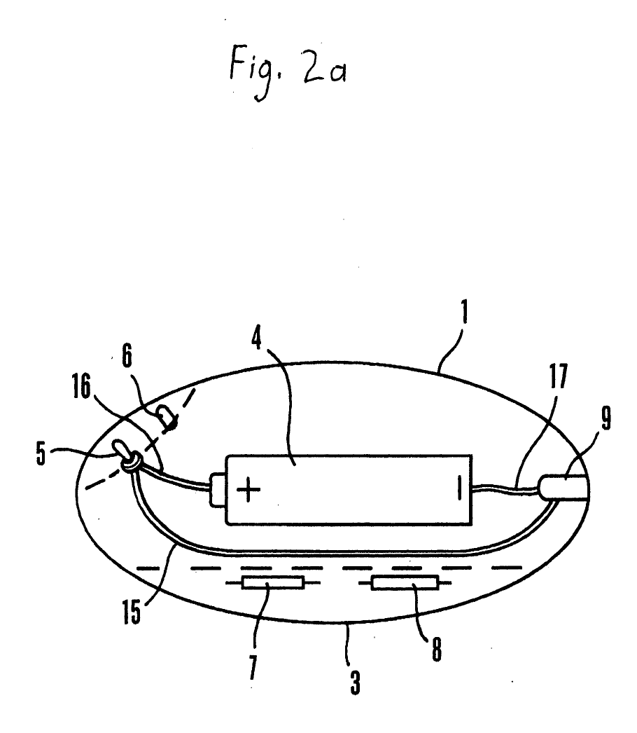

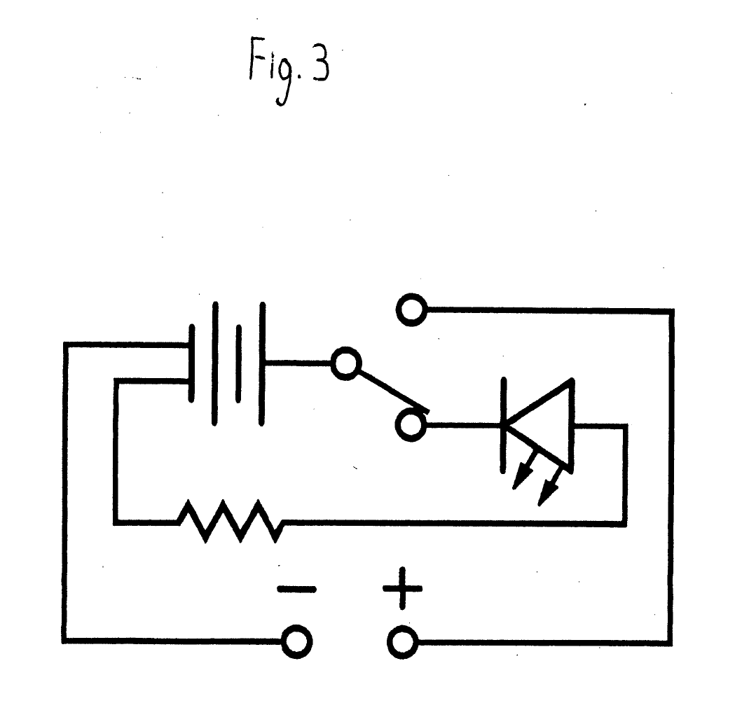

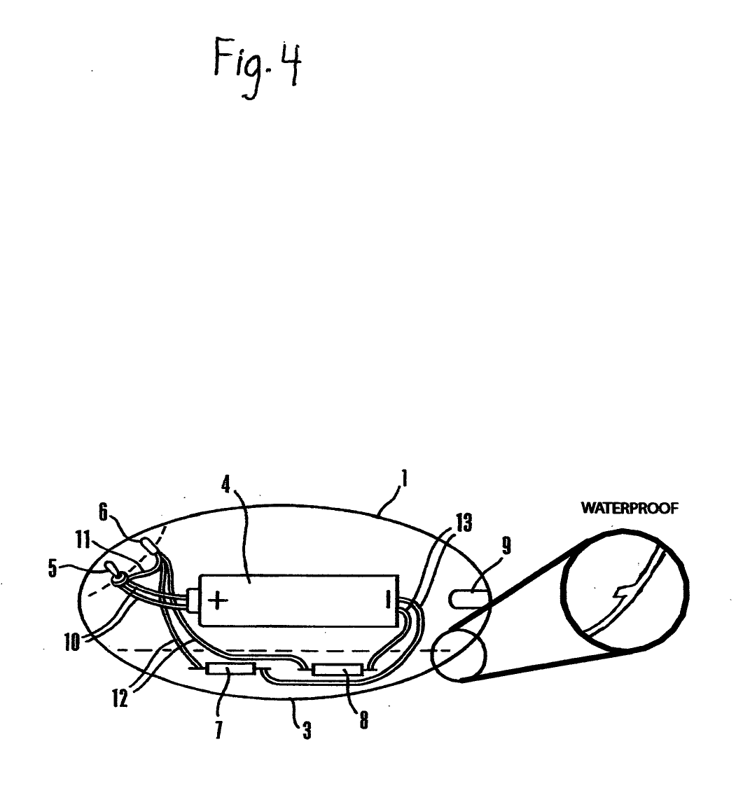

FIG. 2a shows an alternative design for the porcelain body of the present invention utilizing the same components in identical ways as those of FIGS. 1 and 2.

### DETAILED DESCRIPTION OF THE INVENTION

With reference to FIG. 1, the heated simulated rock is designated by the reference numerals 1 for the top, or handheld, portion, and 3 for the bottom, or heated portion. The positive terminals of the batteries 4 are connected by wires 10 to a three position switch 5. In FIG. 1, the three position switch 5 is in position to heat the heated portion 3. Two wires 11 connect the three position switch 5 to a light emitting diode 6, showing the user that the simulated rock is in heating mode and allowing the user awareness of eventual battery wear. Two wires 12 connect the light emitting diode 6 to individual resistors 7 & 8, thereby heating the heated portion 3. Two wires 13 connect the resistors 7 & 8 to the negative terminal of the batteries 4, completing the circuit.

With reference to FIG. 2, the heated simulated rock is designated by the reference numerals 1 for the top, or handheld, portion, and 3 for the bottom, or heated, portion.

Two wires 16 connect the positive terminals of the batteries 4 to the three position switch 5. In FIG. 2, the three position switch 5 is in position to recharge the batteries 4. Two wires 15 connect the three position switch 5 to a DC power plug 9. Two wires 17 connect the DC plug 9 to the negative terminals on the batteries 4 completing the circuit and recharging the batteries 4 when an AC transformer is plugged into the DC plug 9.

Accordingly, an invention has been disclosed in terms of a preferred embodiment thereof which fulfills each of the objects of the present invention as set forth hereinabove and provides a new and useful heated simulated rock for massage therapeutic use.

Various changes, modifications and alterations in the teachings of the present invention may be contemplated by those skilled in the art without departing from the intended spirit and scope thereof.

It is intended, therefore, that the present invention only be limited by the terms of the appended claims.

We claim:

1. A heated simulated rock, comprising: an handheld unheated upper body portion made to resemble a rock and an heated lower portion.

2. The heated simulated rock of claim 1, wherein the lower portion is heated by two resistors powered by custom batteries contained within the simulated rock.

3. The heated simulated rock of claim 1, is made of porcelain and is coated with a waterproofing material.
